(19) United States
(12) Patent Application Publication
(10) Pub. No.: US 2010/0049047 A1
(43) Pub. Date: Feb. 25, 2010

(54) Formation Of An Elastic Image In An Ultrasound System

(76) Inventors: Dong Kuk SHIN, Seoul (KR); Mok Kun Mok JEONG, Seoul (KR)

Correspondence Address: JONES DAY, 222 EAST 41ST ST, NEW YORK, NY, 10017, US

(21) Appl. No.: 12/541,800

(22) Filed: Aug. 14, 2009

(30) Foreign Application Priority Data
Aug. 22, 2008 (KR) 10-2008-0082181

(51) Int. Cl. A61B 8/14

(52) U.S. Cl. 600/443

(57) ABSTRACT

Embodiments for forming an elastic image in an ultrasound system are disclosed. In one embodiment, an ultrasound data acquisition unit is configured to transmit/receive first ultrasound signals to/from a target object to output first ultrasound data and to transmit/receive second ultrasound signals for applying an acoustic radiation force impulse (ARFI) to/from the target object to output second ultrasound data. An image forming unit is configured to form a reference image based on the first ultrasound data and to form an ARFI image based on the second ultrasound data. A processing unit is connected to the ultrasound data acquisition unit and the image forming unit. The processing unit is configured to detect first feature points of the target object in the reference image, to detect second feature points of the target object in the ARFI image and to calculate stress data at each of the first feature points based on the first and second feature points. The processing unit is further configured to form an elastic image based on the stress data.

The present application claims priority from Korean Patent Application No. 10-2008-0082181 filed on Aug. 22, 2008, the entire subject matter of which is incorporated herein by reference.

### TECHNICAL FIELD

The present disclosure relates to ultrasound systems, and more particularly to the formation of an elastic image in an ultrasound system.

### BACKGROUND

Recently, an ultrasound system has been extensively used in the medical field due to its non-invasive and non-destructive nature. Modern high-performance ultrasound imaging systems and techniques are commonly used to produce two dimensional ultrasound images and three-dimensional ultrasound images of internal features of patients.

Generally, the ultrasound image is displayed in a Brightness mode (B-mode) by using reflectivity caused by an acoustic impedance difference between the tissues of a target object. However, if the reflectivity of the target object is hardly different from those of the neighboring tissues such as tumor, cancer or the like, then it is not easy to recognize the target object in the B-mode image. Further, an ultrasound elastic imaging technology has been developed to display an image of the target object by using mechanical characteristics of the target object. Such technology is very helpful for diagnosing lesions such as cancers. The tumor or cancer is relatively stiffer than the neighboring tissues. Thus, when a pressure is uniformly applied, a variation of the tumor or cancer is typically smaller than those of the neighboring tissues.

The elasticity of a tissue is measured by using ultrasound data obtained before and after applying the pressure to the tissue. A compression plate mounted on an ultrasound probe may be used to apply the pressure to the tissue. A user may press the compression plate on the target object, thereby applying the pressure to the tissues of the target object. In such a case, strain data in the tissues may be varied according to the pressure applied by the user. Thus, the video quality of an elastic image may be changed according to the pressure applied to the tissue.

### SUMMARY

Embodiments for forming an elastic image in an ultrasound system are disclosed herein. In one embodiment, by way of non-limiting example, an ultrasound system comprises: an ultrasound data acquisition unit configured to transmit/receive first ultrasound signals to/from a target object to output first ultrasound data and to transmit/receive second ultrasound signals for applying an acoustic radiation force impulse (ARFI) to/from the target object to output second ultrasound data; an image forming unit configured to form a reference image based on the first ultrasound data and to form an ARFI image based on the second ultrasound data; and a processing unit connected to the ultrasound data acquisition unit and the image forming unit, the processing unit being configured to detect first feature points of the target object in the reference image, to detect second feature points of the target object in the ARFI image and to calculate stress data at each of the first feature points based on the first and second feature points, the processing unit being further configured to form an elastic image based on the stress data.

In another embodiment, there is provided a method of forming an elastic image in an ultrasound system, comprising: a) transmitting/receiving by using an ultrasound data acquisition unit within the ultrasound system first ultrasound signals to/from a target object to output first ultrasound data; b) forming by using an image forming unit within the ultrasound system a reference image based on the first ultrasound data; c) detecting by using a processing unit within the ultrasound system first feature points of the target object in the reference image; d) transmitting/receiving by using the ultrasound data acquisition unit within the ultrasound system second ultrasound signals for applying an acoustic radiation force impulse (ARFI) to/from the target object to output second ultrasound data; e) forming by using the image forming unit within the ultrasound system an ARFI image based on the second ultrasound data; f) detecting by using the processing unit within the ultrasound system second feature points of the target object in the ARFI image; g) calculating by using the processing unit within the ultrasound system stress data at each of the first feature points based on the first and second feature points; and h) forming by using the processing unit within the ultrasound system an elastic image based on the stress data.

In yet another embodiment, there is provided a computer readable medium comprising computer executable instructions configured to perform following acts: a) transmitting/receiving first ultrasound signals to/from a target object to output first ultrasound data; b) forming a reference image based on the first ultrasound data; c) detecting first feature points of the target object in the reference image; d) transmitting/receiving second ultrasound signals for applying an acoustic radiation force impulse (ARFI) to/from the target object to output second ultrasound data; e) forming an ARFI image based on the second ultrasound data; f) detecting second feature points of the target object in the ARFI image; g) calculating stress data at each of the first feature points based on the first and second feature points; and h) forming an elastic image based on the stress data.

The Summary is provided to introduce a selection of concepts in a simplified form that are further described below in the Detailed Description. This Summary is not intended to identify key or essential features of the claimed subject matter, nor is it intended to be used in determining the scope of the claimed subject matter.

### BRIEF DESCRIPTION OF THE DRAWINGS

FIG. 1 is a block diagram showing an illustrative embodiment of an ultrasound system.

FIG. 2 is a block diagram showing an illustrative embodiment of an ultrasound data acquisition unit within the ultrasound system as shown in FIG. 1.

FIG. 3 is a block diagram showing an illustrative embodiment of a processing unit within the ultrasound system as shown in FIG. 1.

FIG. 4 is a schematic diagram showing an example of feature points.

### DETAILED DESCRIPTION

A detailed description may be provided with reference to the accompanying drawings. One of ordinary skill in the art may realize that the following description is illustrative only and is not in any way limiting. Other embodiments of the present invention may readily suggest themselves to such skilled persons having the benefit of this disclosure.

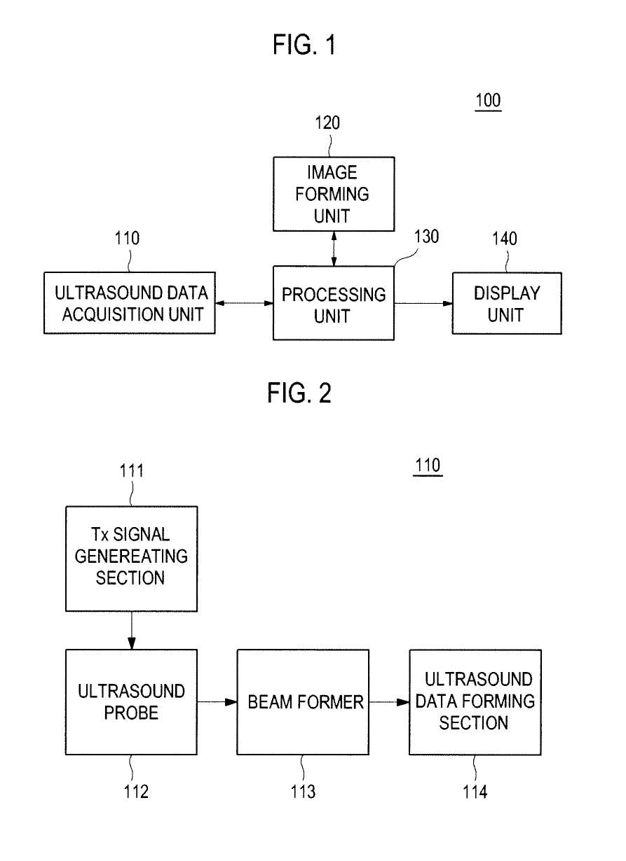

Referring to FIG. 1, an ultrasound system 100 in accordance with an illustrative embodiment is shown. As depicted therein, the ultrasound system 100 may include an ultrasound data acquisition unit 110 configured to transmit/receive ultrasound signals to/from a target object to thereby acquire ultrasound data. The ultrasound data acquisition unit 110 may include a transmit (Tx) signal generating section 111, as shown in FIG. 2.

Referring to FIG. 2, the Tx signal generating section 111 may be operable to generate first Tx signals. The Tx signal generating section 111 may be further operable to generate second Tx signals in consideration of first feature points of the target object in a reference image. The first feature points and the reference image may be defined as below.

The ultrasound data acquisition unit 110 may further include an ultrasound probe 112 containing a plurality of elements for reciprocally converting between ultrasound signals and electrical signals. The ultrasound probe 112 may be operable to transmit first ultrasound signals to the target object in response to the first Tx signals. The ultrasound probe 112 may be further operable to receive echo signals reflected from the target object to thereby output first received signals. The ultrasound probe 112 may be further operable to transmit second ultrasound signals for applying an acoustic radiation force impulse (ARFI) to the target object in response to the second Tx signals so that the compression to the target object can be performed. The second ultrasound signals may be focused on each of the first feature points. The ultrasound probe 112 may be also operable to receive echo signals reflected from the target object to thereby output second received signals.

The ultrasound data acquisition unit 110 may further include a beam former 113. The beam former 113 may be operable to convert the first received signals into first digital signals. The beam former 113 may be further operable to apply delays to the first digital signals in consideration of a distance between the elements and focal points to thereby output first digital receive-focused signals. The beam former 113 may be operable to convert the second received signals into second digital signals. The beam former 113 may be further operable to apply delays to the second digital signals in consideration of a distance between the elements and focal points (i.e., first feature points) to thereby output second digital receive-focused signals.

The ultrasound data acquisition unit 110 may further include an ultrasound data forming section 114. The ultrasound data forming section 114 may be operable to form first ultrasound data based on the first digital receive-focused signals. The ultrasound data forming section 114 may be further operable to form second ultrasound data based on the second digital receive-focused signals.

Referring back to FIG. 1, the ultrasound system 100 may further include an image forming unit 120. The image forming unit 120 may be operable to form the reference image based on the first ultrasound data. The reference image may include one of a brightness mode (B mode) image and a three-dimensional image. The image forming unit 120 may be further operable to an ARFI image based on the second ultrasound data.

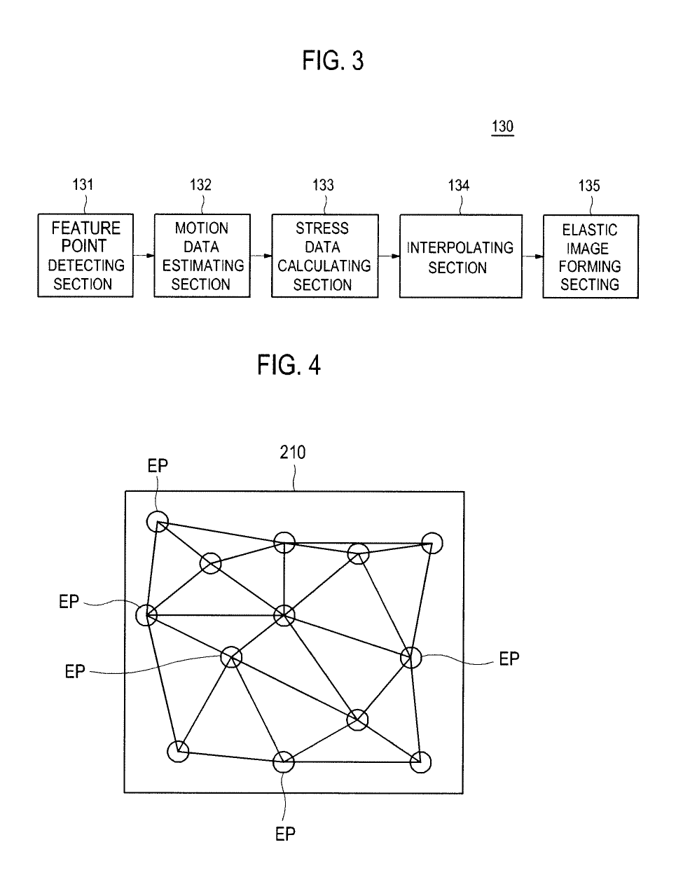

The ultrasound system 100 may further include a processing unit 130 that may be connected to the ultrasound data acquisition unit 110 and the image forming unit 120. The processing unit 130 may include a feature point detecting section 131, as shown in FIG. 3.

Referring to FIG. 3, the feature point detecting section 131 may be operable to detect the first feature points of the target object in the reference image. The feature point detecting section 131 may be further operable to detect second feature points of the target object in the ARFI image. The first and second feature points may include boundary points of the target object. However, the feature points may not be limited thereto. Also, the first and second feature points may be detected based on a variation of brightness determined by using a differential operator. In one embodiment, the feature point detecting section 131 may be configured to detect the feature points by using an edge mask such as Sobel, Prewitt, Robert, Canny and the like. In another embodiment, the feature point detecting section 131 may be configured to detect the feature points based on a difference of eigenvalues using a structure tensor.

The processing unit 130 may further include a motion data estimating section 132. The motion data estimating section 132 may be operable to identify matching points between the first feature points and the second feature points to estimate motion data based on the identified matching points. In one embodiment, the estimation of the motion data may be performed by using various image processing techniques such as an optical flow, a block matching, etc. However, the estimation of the motion data may not be limited thereto.

The processing unit 130 may further include a stress data calculating section 133. The stress data calculating section 133 may be operable to calculate stress data (hereinafter referred to as “first stress data”) of the respective first feature points by using the estimated motion data and an amplitude (or power) of the second ultrasound signals. For example, under the assumption that the target object of a flat shape absorbs ultrasound signals, the stress calculating section 133 may be operable to calculate the stress data (F) of the respective first feature points as in the following equation:

$$F = \frac{{W}_{absorbed}}{c} = \frac{2 α I}{c} \qquad ( 1 )$$

wherein, W_{absorbed} represents power absorbed by the target object at a predetermined spatial position, c represents a sound velocity in the target object, α represents an absorption coefficient of the target object, and I represents a temporal average intensity at a predetermined point.

The processing unit 130 may further include an interpolating section 134. The interpolating section 134 may be operable to interpolate the first stress data to thereby calculate stress data (hereinafter referred to as “second stress data”) corresponding to pixels of an elastic image. In one embodiment, the interpolating section 134 may be operable to detect two adjacent first feature points at each of the first feature points EP, as shown in FIG. 4. The interpolating section 134 may be further operable to interpolate the first stress data at each of the first feature points and the first stress data of the detected two adjacent first feature points to thereby calculate the second stress data corresponding to pixels of the elastic image.

The processing unit 130 may further include an elastic image forming section 135. The elastic image forming section 135 may be operable to form the elastic image based on the second stress data provided from the interpolating section 134.

Referring back to FIG. 1, the ultrasound system 100 may further include a display unit 140 configured to display the elastic image provided from the image processing unit 130. The display unit 140 may be one of a liquid crystal display, a cathode ray tube, a plate display and the like capable of displaying the elastic image. However, the display unit 140 may not be limited thereto. Also, the reference image and the ARFI image may be displayed on the display unit 140.

Although embodiments have been described with reference to a number of illustrative embodiments thereof, it should be understood that numerous other modifications and embodiments can be devised by those skilled in the art that will fall within the spirit and scope of the principles of this disclosure. More particularly, numerous variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

What is claimed is:

1 An ultrasound system, comprising:
an ultrasound data acquisition unit configured to transmit/receive first ultrasound signals to/from a target object to output first ultrasound data and to transmit/receive second ultrasound signals for applying an acoustic radiation force impulse (ARFI) to/from the target object to output second ultrasound data;
an image forming unit configured to form a reference image based on the first ultrasound data and to form an ARFI image based on the second ultrasound data; and
a processing unit connected to the ultrasound data acquisition unit and the image forming unit, the processing unit being configured to detect first feature points of the target object in the reference image, to detect second feature points of the target object in the ARFI image and to calculate stress data at each of the first feature points based on the first and second feature points, the processing unit being further configured to form an elastic image based on the stress data.

2. The ultrasound system of claim 1, wherein the ultrasound data acquisition unit is configured to focus the second ultrasound signals on each of the first feature points.

3. The ultrasound system of claim 1, wherein the processing unit comprises:
a feature point detecting section configured to detect the first feature points of the target object in the reference image and to detect the second feature points of the target object in the ARFI image;
a motion data estimating section configured to identify matching points between the first feature points and the second feature points and to estimate motion data based on the identified matching points;
a stress data calculating section configured to calculate first stress data at each of first feature points based on the estimated motion data;
an interpolating section configured to interpolate the first stress data to calculate second stress data corresponding to pixels of the elastic image; and
an elastic image forming section configured to form the elastic image based on the second stress data.

4. The ultrasound system of claim 3, wherein the interpolating section is configured to detect two adjacent first feature points at each of the first feature points and to interpolate the first stress data at each of the feature points and the first stress data at the detected two adjacent first feature points.

5. The ultrasound system of claim 1, wherein the reference image comprises one of a brightness mode image and a three-dimensional image.

6. A method of forming an elastic image in an ultrasound system, comprising:
a) transmitting/receiving by using an ultrasound data acquisition unit within the ultrasound system first ultrasound signals to/from a target object to output first ultrasound data;
b) forming by using an image forming unit within the ultrasound system a reference image based on the first ultrasound data;
c) detecting by using a processing unit within the ultrasound system first feature points of the target object in the reference image;
d) transmitting/receiving by using the ultrasound data acquisition unit within the ultrasound system second ultrasound signals for applying an acoustic radiation force impulse (ARFI) to/from the target object to output second ultrasound data;
e) forming by using the image forming unit within the ultrasound system an ARFI image based on the second ultrasound data;
f) detecting by using the processing unit within the ultrasound system second feature points of the target object in the ARFI image;
g) calculating by using the processing unit within the ultrasound system stress data at each of the first feature points based on the first and second feature points; and
h) forming by using the processing unit within the ultrasound system an elastic image based on the stress data.

7. The method of claim 6, wherein the step d) comprises:
focusing the second ultrasound signals on each of the first feature points.

8. The method of claim 6, wherein the step g) comprises:
g1) identifying matching points between the first feature points and the second feature points;
g2) estimating motion data based on the identified matching points;
g3) calculating first stress data at each of first feature points based on the estimated motion data; and
g4) interpolating the first stress data to calculate second stress data corresponding to pixels of the elastic image.

9. The method of claim 8, wherein the step g4) comprises:
detecting two adjacent first feature points at each of the first feature points; and
interpolating the first stress data at each of the feature points and the first stress data at the detected two adjacent first feature points to calculate the second stress data.

10. The method of claim 6, wherein the reference image comprises one of a brightness mode image and a three-dimensional image.

11. A computer readable medium comprising computer executable instructions configured to perform following acts:
a) transmitting/receiving first ultrasound signals to/from a target object to output first ultrasound data;
b) forming a reference image based on the first ultrasound data;
c) detecting first feature points of the target object in the reference image;
d) transmitting/receiving second ultrasound signals for applying an acoustic radiation force impulse (ARFI) to/from the target object to output second ultrasound data;
e) forming an ARFI image based on the second ultrasound data;
f) detecting second feature points of the target object in the ARFI image;
g) calculating stress data at each of the first feature points based on the first and second feature points; and
h) forming an elastic image based on the stress data.

12. The computer readable medium of claim 11, wherein the act d) comprises:
focusing the second ultrasound signals on each of the first feature points.

13. The computer readable medium of claim 11, wherein the act g) comprises:
g1) identifying matching points between the first feature points and the second feature points;
g2) estimating motion data based on the identified matching points;
g3) calculating first stress data at each of first feature points based on the estimated motion data; and
g4) interpolating the first stress data to calculate second stress data corresponding to pixels of the elastic image.

14. The computer readable medium of claim 11, wherein the act g4) comprises:
detecting two adjacent first feature points at each of the first feature points; and
interpolating the first stress data at each of the feature points and the first stress data at the detected two adjacent first feature points to calculate the second stress data.

15. The computer readable medium of claim 11, wherein the reference image comprises one of a brightness mode image and a three-dimensional image.
